(19) United States
(12) Patent Application Publication
(10) Pub. No.: US 2013/0309282 A1
(43) Pub. Date: Nov. 21, 2013

(54) HAIRSTYLING AGENT COMPOSITION

(71) Applicant: Kikuboshi Corporation, Tokyo (JP)

(72) Inventor: Miki TAKEHANA, Tokyo (JP)

(73) Assignee: Kikuboshi Corporation, Tokyo (JP)

(21) Appl. No.: 13/950,749

(22) Filed: Jul. 25, 2013

Related U.S. Application Data
(63) Continuation of application No. PCT/JP2011/075050, filed on Oct. 31, 2011.

(30) Foreign Application Priority Data
Jan. 31, 2011 (JP) 2011-017943

(51) Int. Cl. A61K 8/25; A61Q 5/06

(52) U.S. Cl. CPC A61K 8/25; A61Q 5/06
USPC 424/401; 424/70.1; 424/70.13; 424/70.12; 424/70.11; 424/724

(57) ABSTRACT

Provided is a technology that enables setting of a durable hairstyle and moreover, enables easy resetting of a hairstyle. A hairstyling agent composition having a hydrophobic silica and a dispersing liquid for dispersing the hydrophobic silica therein.

### TECHNICAL FIELD

The present invention relates to a hairstyling agent composition.

### BACKGROUND ART

Hairstyling agents (hair dressing agents) can be classified into an agent type that fixes hair with a polymer resin (which will hereinafter be called “polymer resin type”) and an agent type that fixes hair with an oil or fat (which will hereinafter be called “oil or fat type”).

Polymer resin type hairstyling agents can firmly fix hair strands including surroundings thereof with a polymer resin. They can therefore set them into a durable hairstyle (for example, Patent Document 1). When polymer resin type hairstyling agents are used, on the contrary, a hairstyle once set therewith cannot easily be changed into another hairstyle. This means that deformation of the hairstyle once fixed breaks the polymer resin so far used for hair fixing so that the hair should be fixed again by newly adding the hairstyling agent.

When oil or fat type hair styling agents are used, a hairstyle can be set by fixing hair strands and surroundings thereof with a solidified oil or fat. Oils or fats have a property facilitating deformation. By making use of such a property of oils or fats, oil or fat type hair styling agents can freely change the hairstyle fixed with them (for example, they can curl straight hair or straighten curled hair). Even after a hairstyle is set with an oil or fat type hairstyling agent, the hairstyle can be set again easily (for example, Patent Documents 2 and 3). Moreover, when the hairstyle is set again, the hairstyling agent is not necessarily added newly because the surface of the hair has still been covered with the oil or fat. On the contrary, when oil or fat type hairstyling agents are used, they cannot firmly fix hair so that there is a possibility of the hairstyle being broken.

Thus, the polymer resin type hairstyling agents and the oil or fat type hairstyling agents differ largely in characteristics. It is therefore common practice to use these two types of hairstyling agents properly, depending on the situation or desired hairstyle.

### CITATION LIST

### Patent Documents

Patent Document 1: JP-A-2009-96776
Patent Document 2: JP-A-2009-13125
Patent Document 3: JP-A-2008-303178

### SUMMARY OF THE INVENTION

Selection and proper use of these two type hairstyling agents however make a hair setting behavior cumbersome. A technology capable of satisfying both the advantage of the polymer resin type, that is, setting of a hairstyle into a durable one and the advantage of the oil or fat type, that is, easy resetting of a hairstyle is desirable, but these advantages are incompatible with each other. It is therefore difficult to achieve such a technology.

In order to solve the above-mentioned problem, an object of the present invention is to provide a technology capable of setting a durable hairstyle and moreover, resetting the hairstyle easily.

The present invention provides the following hairstyling agent compositions.

[1] A hairstyling agent composition having a hydrophobic silica and a dispersing liquid for dispersing therein the hydrophobic silica.

[2] The hairstyling agent composition as described above in [1], wherein the hydrophobic silica has an average particle size of from 0.1 nm to 20 μm.

[3] The hair styling agent composition as described above in [1] or [2], wherein the dispersing liquid contains

[4] The hairstyling agent composition as described above in any of [1] to [3], wherein the dispersing liquid contains a dispersing aid for enhancing the dispersibility of the hydrophobic silica in the dispersing liquid.

[5] The hairstyling agent composition as described above in [4], wherein the dispersing aid is composed of a polyhydric alcohol and/or a surfactant.

[6] The hairstyling agent composition as described above in [5], wherein the polyhydric alcohol contains at least one selected from a group consisting of dihydric alcohols, condensates of dihydric alcohols, trihydric alcohols, condensates of trihydric alcohols, tetrahydric alcohols, condensates of tetrahydric alcohols, polysaccharides, and compounds of polysaccharides.

[7] The hairstyling agent composition as described above in any of [1] to [6], further containing a lubricant.

[8] The hairstyling agent composition as described above in [7], wherein the lubricant contains at least one selected from a group consisting of a silicone oil, a silicone powder, and hydrogenated polyisobutene.

[9] The hairstyling agent composition as described above in [8], wherein the silicone oil contains at least one selected from a group consisting of dimethyl silicone oil, methyl phenyl silicone oil, cyclic dimethyl silicone oil, methyl hydrogen silicone oil, vinyl dimethicone, dimethylpolysiloxane, methyl phenyl polysiloxane, and polyorganosilsesquioxane.

[10] The hairstyling agent composition as described above in [8], wherein the silicone powder contains at least one selected from a group consisting of trimethylsiloxysilicic acid, methylsiloxane network polymer, crosslinked silicone-network type silicone block copolymer, and crosslinked silicone powder.

[11] The hairstyling agent composition as described above in any of [1] to [10], further having a washing aid.

[12] The hairstyling agent composition as described above in [11], wherein the washing aid contains at least one selected from a group consisting of ethoxy diglycol, ethoxy ethanol, butoxy ethanol, and methoxy ethanol.

[13] The hairstyling agent composition as described above in any of [1] to [12], further having a hair growth promoting component.

[14] The hairstyling agent composition as described above in [13], wherein the hair growth promoting component contains at least one selected from a group consisting of β-glycyrrhetinic acid, dipotassium glycyrrhizate, monoammonium glycyrrhizate, alkaloid from Stephania cepharantha HAYATA (cepharanthine), swertia japonica extract, tinctura capsici, hinokitiol, iodized garlic extract, pyridoxine hydrochloride (Vitamin B6), dl-α-tocopherol (Vitamin E), dl-α-tocopherol acetate (Vitamin E), nicotinic acid amide, benzyl nicotinate, calcium pantothenate, D-pantothenyl alcohol, acetyl pantothenyl ethyl ether, biotin, adenosine, allantoin, isopropyl methylphenol, estradiol, ethinyl estradiol, capronium chloride, benzalkonium chloride, diphenhydramine hydrochloride, Kankohso 301, camphor, salicylic acid, nonanoic acid vanilamide, piroctone olamine, glyceryl pentadecanoate, l-menthol, mononitroguaiacol, resorcin, Sophora Angustifolia root extract, minoxidil, γ-aminobutyric acid, aloe extract, ginkgo extract, benzethonium chloride, mexiletine hydrochloride, Scutellaria Baicalensis root extract, auxin, oxendolone, hypericum extract, seaweed extract, cantharides tincture, diisopropylamine dichloroacetate, cyclosporin, ginger tincture, zinc pyrithione, lansium domesticum extract, calendula extract, cordyceps extract, 2-L-ascorbic acid dl-α-tocopherol phosphoric acid diester potassium salt, peppermint oil, 10-hydroxy-δ-2-decenoic acid, hydrocortisone, rosa rugosa var. plena extract, mini-sasanishiki extract, reishi mushroom extract, polyoxyethylene (20) sorbitan monostearate, rehmanniae radix extract, placenta extract, soybean extract, belamcanda chinensis root extract, chlorella vulgaris extract, hematin porcine, mucopolysaccharide (porcine), Japanese pepper extract, clove extract, cherry leaf extract, sage extract, tilia cordata flower extract, tree paeony extract, and houttuynia cordata extract.

[15] The hairstyling agent composition as described above in [13] or [14], wherein the hair growth promoting component is an oil soluble substance.

The hairstyling agent composition of the present invention makes it possible to set hair into a durable hairstyle and moreover, reset the hairstyle easily.

### MODE FOR CARRYING OUT THE INVENTION

Embodiments of the present invention will hereinafter be described. The present invention is however not limited to the following embodiments, but can be changed, modified or improved without departing from the scope of the invention.

1. Hairstyling agent composition:

The hairstyling agent composition of the present invention contains a hydrophobic silica and a dispersing liquid for dispersing the hydrophobic silica therein.

The hydrophobic silica to be used in the present invention is a substance in particle form. Moreover, the surface of the particle of the hydrophobic silica has hydrophobicity so that the hydrophobic silica to be used in the present invention is easily attached to hair.

When two or more hydrophobic silica particles to be used in the present invention are close to each other, these hydrophobic silica particles are sometimes bound to each other by intermolecular force or electrostatic adhesive force to form an aggregate containing the two or more hydrophobic silica particles.

The dispersing liquid to be used in the invention is a liquid having a property of dispersing therein the hydrophobic silica particles or aggregate of a hydrophobic silica.

It is to be noted that the dispersing liquid to be used in the present invention does not embrace dispersing liquid having a property of turning the hydrophobic silica into a gel and thereby solidifying the entirety of the hair styling agent composition.

The dispersing liquid to be used in the present invention desirably evaporates quickly after it is attached to hair.

When after application of the hairstyling agent composition of the present invention to hair, the dispersing agent disappears from the hair by evaporation or the like, a number of hydrophobic silica particles or a number of aggregates of a hydrophobic silica which have remained after evaporation attach to the hair. When a plurality of hair strands subjected to such a treatment is bundled, the hydrophobic silica particles or aggregates of a hydrophobic silica attached to each of the hair strands bind to each other. In such a manner, a plurality of hair strands can be bound to each other. In short, a hairstyle in which hair strands have been bundled can be obtained by setting with the hairstyling agent of the present invention.

In general, a number of hydrophobic silica particles or a number of aggregates of a hydrophobic silica attached to hair cause further aggregation of these hydrophobic silica particles or aggregates of a hydrophobic silica on the surface of hair, which may result in covering of the surface of hair with aggregates of a hydrophobic silica connected to each other in network form.

Such aggregates of a hydrophobic silica in network form can retain the once formed network form as is by the intermolecular force or electrostatic adhesion force among hydrophobic silica particles. Since such a network form can be retained, the aggregates of a hydrophobic silica in network form surround the individual strands of hair and play a role as a skeleton. As a result, a desired shape of the hair can be retained when the hairstyling agent composition of the present invention is used. When hair is bent and the aggregate of a hydrophobic silica is formed in network form, the bent hair can be retained as is.

When the hair is combed or a hairstyle is changed after set with the hairstyling agent composition of the present invention, the hydrophobic silica particles or aggregates of a hydrophobic silica are released from the bond. This means that a hairstyle obtained by bundling the hair can be combed into individual strands of hair by releasing the hydrophobic silica particles or aggregates of a hydrophobic silica from their bonds. When the hairstyle formed by bending the hair is retained using the aggregates of a hydrophobic silica in network form, by combing the hair it is possible to release the hydrophobic silica particles or aggregates of a hydrophobic silica from their bonds, collapse the skeleton of the network of the aggregates of a hydrophobic silica, and thereby straighten the hair.

Even after the hair bundle is released into individual strands of hair or the hairstyle is changed, it is possible to reset the hairstyle by making use of the residue of the hydrophobic silica particles or aggregates of a hydrophobic silica on the hair. This means that when the hairstyling agent composition of the present invention is applied to hair, the hairstyle once broken can be set again without newly adding the hairstyling agent to the hair.

As the hydrophobic silica or dispersing liquid, those enabling the above-mentioned behavior can be used for the hairstyling agent composition of the present invention.

The hydrophobic silica to be used in the present invention has an average particle size of preferably from 0.1 nm to 20 μm. The hydrophobic silica to be used in the present invention and having an average particle size of from 0.1 nm to 20 μm is likely to cause aggregation on the surface of the hair. Moreover, from the viewpoint of the ease of causing aggregation of the hydrophobic silica particles, the hydrophobic silica to be used in the present invention has an average particle size of preferably from 0.1 nm to 5.0 μm. Further, from the standpoint of the ease of setting the hairstyle and enhancing the retention of the hairstyle, the average particle size is more preferably from 1.0 nm to 1.0 μm and in particular, the average particle size is still more preferably of from 1.0 nm to 100 nm.

The hydrophobic silica to be used in the present invention and having an average particle size of from 0.1 nm to 5.0 μm does not easily precipitate in the dispersing liquid when the hairstyling agent composition is left to stand. Moreover, the hydrophobic silica to be used in the present invention and having an average particle size of from 0.1 nm to 5.0 μm, even if it has precipitated, can be easily dispersed in the dispersing liquid again by vigorously shaking the hairstyling agent composition.

The term “average particle size of the hydrophobic silica” as used herein means an average particle size of primary particles of the hydrophobic silica. The term “average particle size of the hydrophobic silica” herein means a tangential diameter [Feret diameter] of the hydrophobic silica in a specified direction measured on an image taken using a transmission electron microscope (TEM). This measuring method of the average particle size of the hydrophobic silica is similar to that employed by Nippon Aerosil Co., Ltd. for the measurement of the average particle size of its products such as “AEROSIL R812”.

Examples of the hydrophobic silica usable in the present invention include those obtained, for example, by subjecting the hydroxyl group (—OH) which has appeared on the surface of silica particles to a treatment such as dimethylsilylation or trimethylsilylation. The properties of the hydrophobic silica will be described with a treatment method such as dimethylsilylation as an example, but needless to say, this description does not intend to limit the hydrophobic silica to be used in the present invention to those obtained by the treatment method exemplified herein.

Examples of commercially available products of the hydrophobic silica usable in the present invention include “AEROSIL R812” (“AEROSIL R812”, product of EVONIK INDUSTRY), “AEROSIL R812S” (“AEROSIL R812S”, product of EVONIK INDUSTRY), “AEROSIL R8200” (“AEROSIL R8200”, product of EVONIK INDUSTRY), “AEROSIL R805” (“AEROSIL R805”, product of EVONIK INDUSTRY), “VM-2270 Aerogel Fine Particles” (product of Dow Corning), and “HDK H2000” (product of Wacker Asahikasei Silicone).

In the hairstyling agent composition of the present invention, the content of the hydrophobic silica from 0.001 to 5.0 mass % is recommended.

When the content of the hydrophobic silica is 0.001 mass % or greater, an amount of the hydrophobic silica sufficient for setting a hairstyle can be attached to the hair. Moreover, when the content of the hydrophobic silica is 0.001 mass' or greater, the surface of the hair can be covered more easily with the aggregates of a hydrophobic silica in network form. As a result, this makes it easy to set a hairstyle and makes it possible to enhance the retention of the hairstyle thus set.

When the content of the hydrophobic silica is 5.0 mass % or less, the resulting hair styling agent composition of the present invention applied to hair can improve the combing ease of the hair.

The content of the hydrophobic silica in the present invention is more preferably from 0.5 to 1.5 mass'. When the content of the hydrophobic silica is 0.5 mass % or greater, a sufficient amount of hydrophobic silica particles or aggregates of a hydrophobic silica can be attached more surely to the surface of the hair. As a result, the hairstyle can be set more easily and the hairstyle thus set can be made more durable.

Hair has usually an oil or fat film on the surface thereof. When the hydrophobic silica particles or aggregates of the hydrophobic silica are incorporated in this oil or fat film, they become transparent and have an affinity for the hair. When the content of the hydrophobic silica is 1.5 mass % or less, the most part of the hydrophobic silica attached to the hair can be incorporated in the oil or fat film and therefore, the hydrophobic silica particles or aggregates of the hydrophobic silica can be caused to have an affinity for the hair.

The dispersing liquid to be used in the present invention preferably contains a lower alcohol.

The lower alcohol has an advantage of easily dispersing the hydrophobic silica therein. In addition, the lower alcohol has high volatility. When the hairstyling agent composition is applied to hair, the lower alcohol can therefore be evaporated immediately from the hair. By evaporating the lower alcohol from the surface of the hair, the hydrophobic silica particles or aggregates of the hydrophobic silica can be attached to the surface of the hair uniformly, which facilitates setting of a hairstyle.

The term “lower alcohol usable in the present invention” as a dispersing liquid means an alcohol having less carbon atoms. In particular, as the lower alcohol usable in the present invention, alcohols having from 1 to 4 carbon atoms are preferred from the standpoint of high volatility and high fluidity. Of these alcohols having from 1 to 4 carbon atoms, at least one of ethanol, 2-propanol (isopropyl alcohol), methanol, 1-butanol, 2-butanol, isobutanol, and t-butanol is more preferably used in the present invention from the standpoint of high volatility and ability of uniformly dispersing therein the hydrophobic silica. Of these lower alcohols exemplified above, ethanol and 2-propanol are still more preferably used in the present invention from the standpoint of less influence on human bodies.

When the dispersing liquid to be used in the present invention contains water, the content of water is preferably from 0.01 to 22 mass % based on the total amount of the hairstyling agent composition (provided that the total amount of the hairstyling agent composition is 100 mass %) from the standpoint of preventing solidification of the whole hairstyling agent composition which will otherwise occur by the gelation of the hydrophobic silica. When the dispersing liquid does not contain a dispersing aid which will be described later, the content of water in the dispersing liquid is more preferably from 0.01 to 10 masse, still more preferably from 0.01 to 7.0 mass % based on the total amount of the hairstyling agent composition from the standpoint of preventing solidification of the whole hairstyling agent composition which will otherwise occur by the gelation of the hydrophobic silica.

The hairstyling agent composition of the present invention preferably contains a dispersing aid. The dispersing aid usable in the present invention is a substance having a property of enhancing dispersibility of the hydrophobic silica in the dispersing liquid. The hairstyling agent composition of the present invention using a dispersing liquid containing the dispersing aid can prevent solidification of the whole hairstyling agent composition which will otherwise occur by the gelation of the hydrophobic silica.

The dispersing aid usable in the present invention is, for example, a substance having, in the molecular structure thereof, a hydrophilic portion and a hydrophobic portion. The hydrophobic portion of such a dispersing aid can have an affinity for the hydrophobic silica, while the hydrophilic portion can have an affinity for water.

In the hairstyling agent composition of the present invention containing, in the dispersing liquid thereof, a dispersing aid having, in the molecular structure thereof, a hydrophilic portion and a hydrophobic portion, the hydrophilic portion of the dispersing aid can suppress the lower alcohol from having an affinity for water, reacting with water. When the affinity of the lower alcohol or the like for water is suppressed, the lower alcohol can be allowed to have an affinity with the hydrophobic silica, which can make the dispersing liquid suited for dispersing the hydrophobic silica therein. As a result, it suppresses aggregation of the hydrophobic silica, thereby suppressing solidification of the whole hairstyling agent composition.

In the hairstyling agent composition of the present invention having a dispersing liquid containing a dispersing aid, the content of water may be increased. In the hairstyling agent composition of the present invention having a dispersing liquid containing a dispersing aid, the content of water may be increased even to 22 mass % based on the total amount of the hairstyling agent composition (provided that the total amount of the hairstyling agent composition is 100 mass %).

Examples of the dispersing aid usable in the present invention and having, in the molecular structure thereof, a hydrophilic portion and a hydrophobic portion include polyhydric alcohols and surfactants.

When the dispersing aid is contained in the hairstyling agent composition of the present invention, the dispersing aid is composed of preferably a polyhydric alcohol and/or surfactant.

The polyhydric alcohol usable in the present invention as the dispersing aid preferably contains at least one selected from a group consisting of dihydric alcohols, condensates of dihydric alcohols, trihydric alcohols, condensates of trihydric alcohols, tetrahydric alcohols, condensates of tetrahydric alcohols, polysaccharides, and compounds of polysaccharides.

Examples of the dihydric alcohols usable as the dispersing aid in the present invention include ethylene glycol, propylene glycol, butylene glycol, pentanediol, and hexanediol. Examples of the condensates of dihydric alcohols usable as the dispersing aid in the present invention include diethylene glycol, triethylene glycol, polyethylene glycol, propylene glycol, butylene glycol, pentanediol, and hexanediol.

Examples of the trihydric alcohols usable as the dispersing aid in the present invention include glycerin. Examples of the condensates of trihydric alcohols include diglycerin and polyglycerin.

Examples of the tetrahydric alcohols usable as the dispersing aid in the present invention include pentaerythritol. Examples of the condensates of tetrahydric alcohols include dipentaerythritol.

Examples of the polysaccharides usable as the dispersing aid in the present invention include sorbitol, mannitol, maltitol, xylitol, and lactitol. Examples of the compounds of polysaccharides usable as the dispersing aid of the present invention include polyoxyethylene sorbitol and polyoxyethylene sorbitan ester.

These polyhydric alcohols, polysaccharides, and compounds of polysaccharides each have, in the molecular structure thereof, a hydrophilic portion and a hydrophobic portion and can produce a surface activating effect. The hairstyling agent composition of the present invention using, as the dispersing aid, such a polyhydric alcohol, polysaccharide, or compound of a polysaccharide has desirably a total content of the polyhydric alcohol, polysaccharide, or compound of a polysaccharide not greater than 10 mass % (provided that the total amount of the hairstyling agent composition is 100 mass %) in order to avoid sticky feeling of the hair to which the composition has been applied.

Examples of the surfactant usable as the dispersing aid in the present invention include various surfactants such as anionic surfactants, cationic surfactants, amphoteric surfactants, and nonionic surfactants.

Examples of the anionic surfactants usable as the dispersing aid in the present invention include fatty acid soaps (sodium oleates) and α-olefin sulfonates [sodium salts of alkene (C8 to C30) monosulfonates]. Additional examples of the anionic surfactants include compounds having an ionized atomic group bound directly to a nonpolar group [specific examples include soaps, paraffin sulfates, alkylbenzene sulfates, a-sulfonated fatty acid salts, alkyl sulfates, secondary alkyl sulfates, sulfated castor oil salts (turkey red oil)], compounds bound to a nonpolar group through an ether bond (specific examples include polyoxyethylene alkyl sulfates, polyethylene glycol, alkyl ether sulfates, polyethylene glycol, phenol ether sulfates, and sorbitan monooleate sulfates), compounds bound to a nonpolar group through an ester bond (specific examples include monoglyceride sulfates, dialkylsuccinic acid sulfates, polyethylene glycol, ester sulfates, and fatty acid isethionates), compounds bound to a nonpolar group through an amide bond (specific examples include alkanolamide sulfates and taurine derivatives), and compounds bound to a nonpolar group through an amidine bond (specific examples include imidazole sulfates).

Examples of the cationic surfactants usable as the dispersing aid in the present invention include quaternary ammonium salts (specific examples include alkyl trimethylammonium salts, alkyl dimethyl benzylammonium salts, alkyl pyridium salts, and alkyl morpholine salts) and quaternized amides (specific examples include quaternized ethylenediamine amides and quaternized polyethyleneimine amides).

Examples of the amphoteric surfactants usable as the dispersing aid in the present invention include imidazolium betaines (R12 to R18), alkylamino acids (alkyl β-aminopropionic acids), betaines, acetylamino acids (specific examples include acetyl β-aminopropionic acid and acetyl peptides), and alkyl imidazolines.

Examples of the nonionic surfactants usable as the dispersing aid in the present invention include polyoxyethylene sorbitan oleates, sorbitan oleates, and polyoxyethylene stearyl ethers.

When, in the hairstyling agent composition of the present invention, the dispersing liquid contains the dispersing aid, the composition is sometimes attached to the hair with the hydrophobic silica particles or aggregates of a hydrophobic silica being surrounded by the dispersing aid, which however depends on the kind of the substance employed as the dispersing aid. The hydrophobic silica particles or aggregates of a hydrophobic silica attached to the hair in such a manner may be easily rinsed off from the hair upon shampooing.

For example, when the dispersing aid is a substance having, in the molecular structure thereof, a hydrophilic portion and a hydrophobic portion, the hydrophobic silica particles or aggregates of a hydrophobic silica are surrounded by the dispersing aid and the hydrophilic portion of the dispersing aid is exposed from the surface of the hydrophobic silica particles or aggregates of a hydrophobic silica. This hydrophilic portion of the dispersing aid has an affinity for water so that when the hair is shampooed, the hydrophilic silica particles or aggregates of a hydrophobic silica, which have been surrounded by the dispersing aid, can be wetted with water as are and separated from the hair.

Moreover, when such a dispersing aid is used, the hydrophobic silica particles or aggregates of a hydrophobic silica can be rinsed off by using a conventional commercially-available shampoo without using a special detergent.

Examples of the dispersing aid facilitating removal, by rinsing, of the hydrophobic silica include glycerin and propylene glycol. This means that the hairstyling agent composition of the present invention preferably contains at least either one of glycerin and propylene glycol as the dispersing aid from the standpoint of facilitating removal of the hydrophobic silica by rinsing. The hairstyling agent composition of the present invention more preferably contains both glycerin and propylene glycol as the dispersing aid from the standpoint of facilitating removal of the hydrophobic silica further by rinsing.

When the hairstyling agent composition of the present invention uses the dispersing aid, using 1,2-hexanediol, caprylyl glycol, 1,10-decanediol, or decylene glycol is preferred from the standpoint that it considerably improves combing ease of hair and removal of the hydrophobic silica by rinsing. For the hairstyling agent composition of the present invention, the above-mentioned dispersing aids such as 1,2-hexanediol may be used either singly or in combination of two or more. In the hairstyling agent composition of the present invention, the dispersing aid containing both 1,2-hexanediol and caprylyl glycol is more preferred from the standpoint that it further improves both combing ease of hair and removal of the hydrophobic silica by rinsing.

Even if the dispersing aid having a property of facilitating removal, by rinsing, of the hydrophobic silica is not used, the hydrophobic silica particles or aggregates of a hydrophobic silica can be rinsed off easily from the hair by using a washing aid.

As the washing aid usable in the present invention, the hairstyling agent composition preferably contains at least one selected from a group consisting of ethoxy diglycol, ethoxy ethanol, butoxy ethanol, and methoxy ethanol. When such a substance is used as the washing aid, it becomes possible to easily wash away the hydrophobic silica from the hair while favorably maintaining the ability to facilitate setting of hairstyles and the retention power of hairstyles (setting power of hairstyle).

In the hairstyling agent composition of the present invention containing ethoxy diglycol, ethoxy ethanol, butoxy ethanol, and/or methoxy ethanol as the washing aid, a total content of these washing aids is preferably from 0.1 to 0.2 mass %, more preferably from 0.3 to 0.4 mass %, most preferably 0.5 mass %, provided that the total amount of the hairstyling agent composition is 100 mass %. The hairstyling agent composition having a total content of these washing aids within a range of from 0.5 to 1.0 mass % can surely maintain the ability to facilitate setting of hairstyles and the retention power of hairstyles (setting power of hairstyles) in a better condition.

The hairstyling agent composition preferably contains a lubricant from the standpoint of facilitating smooth combing of the hair. The smooth combing of the hair can be achieved by weakening the aggregation power of the hydrophobic silica that binds hair strands to each other. As the lubricant, therefore, a substance capable of reducing the aggregation power of hydrophobic silica (power for binding the hydrophobic silica particles to each other) is recommended.

With regards to the lubricant usable in the present invention, the degree of reducing the aggregation power of the hydrophobic silica differs, depending on the kind of a substance used as the lubricant. It is therefore possible to regulate the aggregation power of the hydrophobic silica and thereby enhance or weaken the retention power of hairstyles (setting power of hairstyles) (refer to, for example, the evaluation in “setting power of hairstyle” of Examples containing various lubricants, shown in Table 1) by selecting the kind of the lubricant as needed.

As the lubricant usable in the present invention, using at least one selected from a group consisting of silicone oils, silicone powders, and hydrogenated polybutene is preferred. Silicone oils, silicone powders, and hydrogenated polybutene are particularly suited for use in the present invention because they have an affinity for hair and in addition, they have little risk of harming human bodies.

Examples of the silicone oils usable in the present invention include those containing at least one selected from a group consisting of dimethyl silicone oils, methyl phenyl silicone oils, cyclic dimethyl silicone oils, methyl hydrogen silicone oils, vinyl dimethicone, dimethylpolysiloxane, methyl phenyl polysiloxane, and polyorganosilsesquioxane.

When the silicone oil is used as the lubricant in the present invention, using a volatile silicone oil as the silicone oil is more preferred because it can fully draw out the effect of the hydrophobic silica attached to the hair. As this volatile silicone oil, a substance having lower volatility than the dispersing liquid is still more preferred.

In the case of the silicone oil having lower volatility than the dispersing liquid, after the hairstyling agent composition is attached to hair, the dispersing liquid evaporates to leave the hydrophobic silica and the volatile silicone oil on the surface of the hair in the first stage and then, the volatile silicone oil evaporates to leave only the hydrophobic silica on the hair in the second stage. In the first stage, combing ease of the hair is improved by the action of the volatile silicone oil, resulting in easy setting of a hairstyle. In the second stage, the volatile silicone oil which has weakened the bond among hydrophobic silica particles disappears and only the hydrophobic silica remains on the surface of the hair so that the binding power among the hydrophobic silica particles increases, resulting in enhanced retention (setting power of hairstyles) of hairstyles.

In short, using the volatile silicone oil as the lubricant in the present invention enables easy setting of hairstyles and makes the hairstyles more durable.

When the volatile silicone oil is used in the present invention, the content of the volatile silicone oil is preferably 30 mass % or less. The hairstyling agent composition having a 30 mass % or less content of the volatile silicone oil can be attached to hair more easily. Moreover, the hairstyling agent composition having the 30 mass % or less content of the volatile silicone oil allows rapid evaporation of the volatile silicone oil from the surface of the hair, making it possible to prevent the hair from becoming sticky and heavy. The content of the volatile silicone oil is more preferably 15 mass % or less because it can surely prevent the hair from becoming sticky and at the same time, it can surely prevent the hair from becoming heavy.

Examples of the dimethyl silicone oils usable in the present invention include methylpolysiloxanes (such as octamethyltrisiloxane, decamethyltetrasiloxane, and tetradecamethylhexasiloxane), tetramethyldisiloxane, and hexamethyldisiloxane. When a lower alcohol is used as the dispersing liquid, it is preferred to use, among the above-described ones, octamethyltrisiloxane, decamethyltetrasiloxane, tetramethylsiloxane, methylpolysiloxane, ethyltrisiloxane, caprylyl methicone, disiloxane, dimethicone, methyl trimethicone or hexamethyldisiloxane in the present invention because the above exemplified one is a volatile silicone oil having lower volatility than the lower alcohol.

As the methyl phenyl silicone oil usable in the present invention, when a lower alcohol is used as the dispersing liquid, using methyl phenyl polysiloxanes (diphenyl dimethicone, phenyl dimethicone, and phenyltridimethicone) is preferred because they are volatile silicone oils having lower volatility than the lower alcohol.

Examples of the cyclic dimethyl silicone oils usable in the present invention include octamethyl cyclotetrasiloxane (cyclotetrasiloxane), decamethyl cyclopentasiloxane (cyclopentasiloxane, cyclomethicone), dodecamethyl cyclohexasiloxane (cyclohexasiloxane), and methyl cyclopolysiloxane (cyclomethicone). When a lower alcohol is used as the dispersing liquid, cyclotetrasiloxane or decamethyl cyclopentasiloxane (cyclopentasiloxane, cyclomethicone) is preferred in the present invention because it is a volatile silicone oil having lower volatility than the lower alcohol.

Examples of the methyl hydrogen silicone oils usable in the present invention include methyl hydrogen polysiloxane.

When the silicone powder is used as the lubricant in the present invention, trimethylsiloxysilicic acid, methyl siloxane network polymer, crosslinked silicone powder, crosslinked silicone/network silicone block copolymer, polymethylsilsesquioxane, polyphenylsilsesquioxane, (diphenyl dimethicone/vinyldiphenyl dimethicone/silsesquioxane) crosspolymer and the like can be used as the silicone powder.

The hairstyling agent composition preferably contains a hair growth promoting component because it can promote hair growth as well as setting hairstyles. Setting of hairstyles is conducted almost every day in the daily life, but it is not intentionally conducted as one of routine duties. When the hairstyling agent composition contains a hair growth promoting component, it is therefore possible to conduct hair growth promoting treatment every day without encouraging yourselves so as not to forget this hair growth treatment.

The hair growth promoting component usable in the present invention preferably contains at least one selected from a group consisting of β-glycyrrhetinic acid, dipotassium glycyrrhizate, monoammonium glycyrrhizate, alkaloid from Stephania cepharantha HAYATA (cepharanthine), swertia japonica extract, tinctura capsici, hinokitiol, iodized garlic extract, pyridoxine hydrochloride (Vitamin B6), dl-α-tocopherol (Vitamin E), dl-α-tocopherol acetate (Vitamin E), nicotinic acid amide, benzyl nicotinate, calcium pantothenate, D-pantothenyl alcohol, acetyl pantothenyl ethyl ether, biotin, adenosine, allantoin, isopropyl methylphenol, estradiol, ethinyl estradiol, capronium chloride, benzalkonium chloride, diphenhydramine hydrochloride, Kankohso 301, camphor, salicylic acid, nonanoic acid vanilamide, piroctone olamine, glyceryl pentadecanoate, l-menthol, mononitroguaiacol, resorcin, Sophora Angustifolia root extract, minoxidil, γ-aminobutyric acid, aloe extract, ginkgo extract, benzethonium chloride, mexiletine hydrochloride, Scutellaria Baicalensis root extract, auxin, oxendolone, hypericum extract, seaweed extract, cantharides tincture, diisopropylamine dichloroacetate, cyclosporin, ginger tincture, zinc pyrithione, lansium domesticum extract, calendula extract, cordyceps extract, 2-L-ascorbic acid dl-α-tocopherol phosphoric acid diester potassium salt, peppermint oil, 10-hydroxy-δ-2-decenoic acid, hydrocortisone, rosa rugosa var. plena extract, mini-sasanishiki extract, reishi mushroom extract, polyoxyethylene (20) sorbitan monostearate, rehmanniae radix extract, placenta extract, soybean extract, belamcanda chinensis root extract, chlorella vulgaris extract, hematin porcine, mucopolysaccharide (porcine), Japanese pepper extract, clove extract, cherry leaf extract, sage extract, tilia cordata flower extract, tree paeony extract, and houttuynia cordata extract.

The hair growth promoting component usable in the present invention is preferably an oil soluble substance from the standpoint of dissolving the hair growth promoting component in the dispersing liquid or uniformly dispersing the hair growth promoting component in the dispersing liquid.

2. Using method of hairstyling agent composition:

The hairstyling agent composition of the present invention is preferably attached to the hair by spraying. In such a manner, the hairstyling agent composition can be sprayed to the hair while dispersing the hydrophobic silica in fine droplets of the dispersing liquid, in other words, while closing the hydrophobic silica in the dispersing liquid. When the hydrophobic silica in powder form is used as is, on the other hand, the particles of the hydrophobic silica float in the vicinity when the hairstyle agent composition is attached to the hair and as a result, a large amount of the particles of the hydrophobic silica is lost without being attached to the hair. The hairstyling agent composition of the present invention can be attached to the hair while closing the hydrophobic silica in the dispersing liquid so that loss of the hydrophobic silica can be reduced. In addition, the actual amount of the hydrophobic silica attached to the hair can therefore be estimated precisely based on the sprayed amount of the hairstyling agent composition.

Moreover, when the hairstyling agent composition is attached to the hair by spraying, fine droplets of the dispersing liquid having the hydrophobic silica dispersed therein can be attached uniformly to the surface of the hair so that aggregates of the hydrophobic silica connected to each other in network form can easily be formed on the surface of the hair. As a result, the effect derived from the aggregates of the hydrophobic silica in network form can be easily produced. This means that by spraying the hairstyling agent composition, hairstyles can be set easily and retention power of hairstyles (setting power of hairstyles) can be enhanced further.

For example, when a stirring ball is placed in a vessel for housing the hairstyling agent composition therein, even the hydrophobic silica which has precipitated in the dispersing liquid can be dispersed rapidly in the dispersing liquid by shaking the vessel to stir the liquid with the stirring ball. This means that by using a stirring ball, the hairstyling agent composition can be made ready for use rapidly even when it is used after storage for a long period of time.

### EXAMPLES

The present invention will hereinafter be described more specifically based on Examples, but the present invention is not limited to or by these Examples.

### (1) Preparation of Hairstyling Agent Composition

The following materials were used for the preparation of hairstyling agent compositions in Examples and Comparative Examples.

(a) Hydrophobic silica:
AEROSIL R812 (“AEROSIL R812”, product of EVONIK, average particle size: 7 nm)
AEROSIL R812S (“AEROSIL R812S”, product of EVONIK, average particle size: 7 nm)
AEROSIL R805 (“AEROSIL R805”, product of EVONIK, average particle size: 12 nm)
AEROSIL R8200 (“AEROSIL R8200”, product of EVONIK, average particle size: 12 nm)
(b) Hydrophilic silica:
“SUNSPHERE NP30” (product of Asahi Glass, particle size: from 3.0 to 5.0 μm)
(c) Dispersing liquid:

Used was a dispersing liquid obtained by adding, to ethanol (Japan Alcohol, general, synthetic, 95% alcohol) or a mixture obtained by adding a predetermined amount of purified water to ethanol (Japan Alcohol, general, synthetic, alcohol), glycerin (trade name: Cosmetic Grade Concentrated Glycerin, product of Kao Corporation), propylene glycol (PG) (trade name: Propylene Glycol (Japanese Pharmacopoeia), product of Adeka), sorbitan sesquioleate (trade name: “Rheodol AO-15V”, product of Kao Corporation), and a mixture of 1,2-hexanediiol and caprylyl glycol (trade name: “Symdiol 68”, product of Symrise AG) as a dispersing aid.

### (d) Lubricant:

Decamethyl cyclopentasiloxane (“TSF405”, product of Momentive PWJ)
Vinyl dimethicone (“KSP-105”, product of Shin-etsu Chemical Industry)
Hydrogenated polyisobutene (“IP Solvent 1620”, product of Idemitsu Petrochemical)

### (e) Washing aid:

Ethoxy diglycol (“Ethyl diglycol”, product of Daicel Corporation)

### Examples 1 to 46, Referential Example 1, Comparative Examples 1 to 5

Hairstyling agent compositions of Examples 1 to 46, Referential Example 1, and Comparative Examples 1 to 5 were prepared by mixing the above-mentioned materials into compositions as shown in Tables 1 to 5 [the content (mass %) of each of the materials in Table 1 to 5 was based on the total amount of the hairstyling agent composition (provided that the total amount of the hairstyling agent composition was 100 mass %)].

TABLE 1

| Dispersing liquid | | |
|---|---|---|
| | Dispersing aid | Lubricant |

| | Silica | | | | | Propylene | Sorbitan | Decamethyl | Vinyl | |
|---|---|---|---|---|---|---|---|---|---|---|
| | | Content | Ethanol | Water | Glycerin | glycol | sesqui- | cyclopenta- | dimethi- | Hydrogenated |
| | | (mass | (mass | (mass | (mass | (PG) | oleate | siloxane | cone | polyisobutene |
| | Trade name | %) | %) | %) | %) | (mass %) | (mass %) | (mass %) | (mass %) | (mass %) |
| Example 1 | AEROSIL R812 | 1.00 | 92.81 | 6.19 | 0.00 | 0.00 | 0.00 | 0.00 | 0.00 | 0.00 |
| Example 2 | AEROSIL R812 | 1.00 | 46.41 | 3.09 | 0.00 | 0.00 | 0.00 | 49.50 | 0.00 | 0.00 |
| Example 3 | AEROSIL R812 | 1.00 | 55.69 | 3.71 | 0.00 | 0.00 | 0.00 | 39.60 | 0.00 | 0.00 |
| Example 4 | AEROSIL R812 | 1.00 | 64.97 | 4.33 | 0.00 | 0.00 | 0.00 | 29.70 | 0.00 | 0.00 |
| Example 5 | AEROSIL R812 | 1.00 | 70.05 | 4.70 | 0.00 | 0.00 | 0.00 | 24.25 | 0.00 | 0.00 |
| Example 6 | AEROSIL R812 | 1.50 | 69.37 | 4.63 | 0.00 | 0.00 | 0.00 | 24.50 | 0.00 | 0.00 |
| Example 7 | AEROSIL R812 | 1.00 | 27.84 | 1.86 | 0.00 | 0.00 | 0.00 | 0.00 | 0.00 | 69.30 |
| Example 8 | AEROSIL R812 | 1.00 | 37.12 | 2.48 | 0.00 | 0.00 | 0.00 | 0.00 | 0.00 | 59.40 |
| Example 9 | AEROSIL R812 | 1.00 | 46.41 | 3.09 | 0.00 | 0.00 | 0.00 | 0.00 | 0.00 | 49.50 |
| Example 10 | AEROSIL R812 | 1.00 | 55.69 | 3.71 | 0.00 | 0.00 | 0.00 | 0.00 | 0.00 | 39.60 |
| Example 11 | AEROSIL R812 | 1.00 | 64.97 | 4.33 | 0.00 | 0.00 | 0.00 | 0.00 | 0.00 | 29.70 |

| | Washing aid | Evaluation of performance | | | | |
|---|---|---|---|---|---|---|
| | Ethoxy | Solidified | Standing | Setting | | |
| | diglycol | or not | degree of | power of | Combing | Removal |
| | (mass %) | solidified | hair | hairstyle | ease | by rinsing |
| Example 1 | 0.00 | Not solidified | Excellent | Excellent (+) | Fair | Fair |
| Example 2 | 0.00 | Not solidified | Fair | Good | Good | Fair |
| Example 3 | 0.00 | Not solidified | Fair | Good | Good | Fair |
| Example 4 | 0.00 | Not solidified | Excellent | Excellent (+) | Fair | Fair |
| Example 5 | 0.00 | Not solidified | Excellent | Excellent (+) | Fair | Fair |
| Example 6 | 0.00 | Not solidified | Excellent | Excellent (+) | Fair | Fair |
| Example 7 | 0.00 | Not solidified | Fair | Good | Excellent | Fair |
| Example 8 | 0.00 | Not solidified | Fair | Good | Good | Fair |
| Example 9 | 0.00 | Not solidified | Fair | Good | Good | Fair |
| Example 10 | 0.00 | Not solidified | Fair | Good | Good | Fair |
| Example 11 | 0.00 | Not solidified | Excellent | Excellent (+) | Fair | Fair |

TABLE 2

| | |
|---|---|
| Dispersing liquid | Lubricant |

| | | | | | Dispersing aid | | | Deca- | | Hydro- |
|---|---|---|---|---|---|---|---|---|---|---|
| | Silica | | | | | Propylene | Sorbitan | methyl | Vinyl | genated |
| | | Content | Ethanol | Water | Glycerin | glycol | sesqui- | cyclopenta- | dimethi- | polyiso- |
| | | (mass | (mass | (mass | (mass | (PG) | oleate | siloxane | cone | butene |
| | Trade name | %) | %) | %) | %) | (mass %) | (mass %) | (mass %) | (mass %) | (mass %) |
| Example 12 | AEROSIL R812 | 0.75 | 64.95 | 4.30 | 0.00 | 0.00 | 0.00 | 0.00 | 0.00 | 30.00 |
| Example 13 | AEROSIL R812 | 0.50 | 65.06 | 4.34 | 0.00 | 0.00 | 0.00 | 0.00 | 0.00 | 30.10 |
| Example 14 | AEROSIL R812 | 0.25 | 65.16 | 4.34 | 0.00 | 0.00 | 0.00 | 0.00 | 0.00 | 30.25 |
| Example 15 | AEROSIL R812 | 1.50 | 64.69 | 4.31 | 0.00 | 0.00 | 0.00 | 0.00 | 0.00 | 29.50 |
| Example 16 | AEROSIL R812 | 1.00 | 67.50 | 4.50 | 0.00 | 0.00 | 0.00 | 12.00 | 0.00 | 15.00 |
| Example 17 | AEROSIL R812 | 1.00 | 92.72 | 6.18 | 0.00 | 0.00 | 0.00 | 0.00 | 0.10 | 0.00 |
| Example 18 | AEROSIL R812S | 1.00 | 92.81 | 6.19 | 0.00 | 0.00 | 0.00 | 0.00 | 0.00 | 0.00 |
| Example 19 | AEROSIL R805 | 1.00 | 92.81 | 6.19 | 0.00 | 0.00 | 0.00 | 0.00 | 0.00 | 0.00 |
| Example 20 | AEROSIL R8200 | 1.00 | 92.81 | 6.19 | 0.00 | 0.00 | 0.00 | 0.00 | 0.00 | 0.00 |

| | Washing aid | Evaluation of performance | | | | |
|---|---|---|---|---|---|---|
| | Ethoxy | Solidified | Standing | Setting | | |
| | diglycol | or not | degree of | power of | Combing | Removal |
| | (mass %) | solidified | hair | hairstyle | ease | by rinsing |
| Example 12 | 0.00 | Not solidified | Fair | Good | Good | Fair |
| Example 13 | 0.00 | Not solidified | Fair | Fair | Good | Fair |
| Example 14 | 0.00 | Not solidified | Fair | Fair | Good | Fair |
| Example 15 | 0.00 | Not solidified | Excellent | Excellent (+) | Bad | Fair |
| Example 16 | 0.00 | Not solidified | Good | Excellent | Fair | Fair |
| Example 17 | 0.00 | Not solidified | Excellent | Excellent (+) | Fair | Fair |
| Example 18 | 0.00 | Not solidified | Good | Excellent | Fair | Fair |
| Example 19 | 0.00 | Not solidified | Good | Excellent | Fair | Fair |
| Example 20 | 0.00 | Not solidified | Good | Excellent | Fair | Fair |

TABLE 3

| Dispersing liquid | | |
|---|---|---|
| | Dispersing aid | Lubricant |

| | Silica | | | | | Propylene | Sorbitan | Decamethyl | Vinyl | |
|---|---|---|---|---|---|---|---|---|---|---|
| | | Content | Ethanol | Water | Glycerin | glycol | sesqui- | cyclopenta- | dimethi- | Hydrogenated |
| | | (mass | (mass | (mass | (mass | (PG) | oleate | siloxane | cone | polyisobutene |
| | Trade name | %) | %) | %) | %) | (mass %) | (mass %) | (mass %) | (mass %) | (mass %) |
| Example 21 | AEROSIL R812 | 1.00 | 69.39 | 4.61 | 4.00 | 6.00 | 0.00 | 15.00 | 0.00 | 0.00 |
| Example 22 | AEROSIL R812 | 1.00 | 72.25 | 4.75 | 1.00 | 6.00 | 0.00 | 15.00 | 0.00 | 0.00 |
| Example 23 | AEROSIL R812 | 1.00 | 66.58 | 4.42 | 7.00 | 6.00 | 0.00 | 15.00 | 0.00 | 0.00 |
| Example 24 | AEROSIL R812 | 1.00 | 75.02 | 4.98 | 4.00 | 10.00 | 0.00 | 5.00 | 0.00 | 0.00 |
| Example 25 | AEROSIL R812 | 1.00 | 66.09 | 4.41 | 0.00 | 0.00 | 5.00 | 23.50 | 0.00 | 0.00 |
| Example 26 | AEROSIL R812 | 1.00 | 66.09 | 9.41 | 0.00 | 0.00 | 0.00 | 23.50 | 0.00 | 0.00 |
| Example 27 | AEROSIL R812 | 1.00 | 66.11 | 9.39 | 2.50 | 6.00 | 0.00 | 15.00 | 0.00 | 0.00 |
| Example 28 | AEROSIL R812 | 1.00 | 61.42 | 14.08 | 2.50 | 6.00 | 0.00 | 15.00 | 0.00 | 0.00 |
| Example 29 | AEROSIL R812 | 1.00 | 56.75 | 18.75 | 2.50 | 6.00 | 0.00 | 15.00 | 0.00 | 0.00 |
| Example 30 | AEROSIL R812 | 1.00 | 64.22 | 6.28 | 0.00 | 0.00 | 5.00 | 23.50 | 0.00 | 0.00 |
| Example 31 | AEROSIL R812 | 1.00 | 62.34 | 8.16 | 0.00 | 0.00 | 5.00 | 23.50 | 0.00 | 0.00 |
| Example 32 | AEROSIL R812 | 1.00 | 60.47 | 10.03 | 0.00 | 0.00 | 5.00 | 23.50 | 0.00 | 0.00 |

| | Washing aid | Evaluation of performance | | | | |
|---|---|---|---|---|---|---|
| | Ethoxy | Solidified | Standing | Setting | | |
| | diglycol | or not | degree of | power of | Combing | Removal |
| | (mass %) | solidified | hair | hair style | ease | by rinsing |
| Example 21 | 0.00 | Not solidified | Fair | Fair | Good | Good |
| Example 22 | 0.00 | Not solidified | Good | Fair | Fair | Good |
| Example 23 | 0.00 | Not solidified | Fair | Fair | Good | Good |
| Example 24 | 0.00 | Not solidified | Fair | Fair | Good | Good |
| Example 25 | 0.00 | Not solidified | Fair | Fair | Good | Fair |
| Example 26 | 0.00 | Not solidified | Fair | Fair | Good | Fair |
| Example 27 | 0.00 | Not solidified | Fair | Fair | Good | Good |
| Example 28 | 0.00 | Not solidified | Fair | Fair | Good | Good |
| Example 29 | 0.00 | Not solidified | Fair | Fair | Good | Good |
| Example 30 | 0.00 | Not solidified | Fair | Fair | Good | Fair |
| Example 31 | 0.00 | Not solidified | Fair | Fair | Good | Fair |
| Example 32 | 0.00 | Not solidified | Fair | Fair | Good | Fair |

TABLE 4

| | | | Dispersing liquid | | | | | | |
|---|---|---|---|---|---|---|---|---|---|
| | | | | | Dispersing aid | | | | |
| | | | | | | Propylene | | | |
| | Silica | | | | | glycol | Sorbitan | 1,2- | caprylyl |
| | | Content | Ethanol | Water | Glycerin | (PG) | sesquioleate | Hexanediol | glycol |
| | Trade name | (mass %) | (mass %) | (mass %) | (mass %) | (mass %) | (mass %) | (mass %) | (mass %) |
| Example 33 | AEROSIL R812 | 1.00 | 74.08 | 4.92 | 2.00 | 3.00 | 0.00 | 0.00 | 0.00 |
| Example 34 | AEROSIL R812 | 1.00 | 70.80 | 4.70 | 2.50 | 6.00 | 0.00 | 0.00 | 0.00 |
| Example 35 | AEROSIL R812 | 1.00 | 64.25 | 6.25 | 2.50 | 6.00 | 5.00 | 0.00 | 0.00 |
| Example 36 | AEROSIL R812 | 1.00 | 62.48 | 8.02 | 2.50 | 6.00 | 5.00 | 0.00 | 0.00 |
| Example 37 | AEROSIL R812 | 1.00 | 60.48 | 10.02 | 2.50 | 6.00 | 5.00 | 0.00 | 0.00 |
| Example 38 | AEROSIL R812 | 1.50 | 70.30 | 4.70 | 2.50 | 6.00 | 0.00 | 0.00 | 0.00 |
| Example 39 | AEROSIL R812 | 2.00 | 69.86 | 4.64 | 2.50 | 6.00 | 0.00 | 0.00 | 0.00 |
| Example 40 | AEROSIL R812 | 3.00 | 68.75 | 4.75 | 2.50 | 6.00 | 0.00 | 0.00 | 0.00 |
| Example 41 | AEROSIL R812 | 5.00 | 67.05 | 4.45 | 2.50 | 6.00 | 0.00 | 0.00 | 0.00 |
| Example 42 | AEROSIL R812 | 1.00 | 70.14 | 4.66 | 2.50 | 6.00 | 0.00 | 0.00 | 0.00 |
| Example 43 | AEROSIL R812 | 1.00 | 70.33 | 4.67 | 2.50 | 6.00 | 0.00 | 0.00 | 0.00 |
| Example 44 | AEROSIL R812 | 1.00 | 70.52 | 4.68 | 2.50 | 6.00 | 0.00 | 0.00 | 0.00 |
| Example 45 | AEROSIL R812 | 1.00 | 70.70 | 4.70 | 2.50 | 6.00 | 0.00 | 0.00 | 0.00 |
| Example 46 | AEROSIL R812 | 1.00 | 70.30 | 4.70 | 2.50 | 6.00 | 0.00 | 0.25 | 0.25 |
| | Lubricant | | | Washing | Evaluation of performance | | | | |
| | Decamethyl | | | aid | | | Setting | | |
| | cyclopenta- | Vinyl | Hydrogenated | Ethoxy | Solidified | Standing | power | | Removal |
| | siloxane | dimethicone | polyisobutene | diglycol | or not | degree | of hair | Combing | by |
| | (mass %) | (mass %) | (mass %) | (mass %) | solidified | of hair | style | ease | rinsing |
| Example 33 | 15.00 | 0.00 | 0.00 | 0.00 | Not | Excellent | Excellent | Good | Good |
| | | | | | solidified | | | | |
| Example 34 | 15.00 | 0.00 | 0.00 | 0.00 | Not | Excellent | Excellent | Good | Good |
| | | | | | solidified | | | | |
| Example 35 | 15.00 | 0.00 | 0.00 | 0.00 | Not | Fair | Fair | Good | Good |
| | | | | | solidified | | | | |
| Example 36 | 15.00 | 0.00 | 0.00 | 0.00 | Not | Fair | Fair | Good | Good |
| | | | | | solidified | | | | |
| Example 37 | 15.00 | 0.00 | 0.00 | 0.00 | Not | Fair | Fair | Good | Good |
| | | | | | solidified | | | | |
| Example 38 | 15.00 | 0.00 | 0.00 | 0.00 | Not | Excellent | Excellent | Good | Good |
| | | | | | solidified | | | | |
| Example 39 | 15.00 | 0.00 | 0.00 | 0.00 | Not | Excellent | Excellent | Good | Good |
| | | | | | solidified | | | | |
| Example 40 | 15.00 | 0.00 | 0.00 | 0.00 | Not | Excellent | Excellent | Good | Good |
| | | | | | solidified | | | | |
| Example 41 | 15.00 | 0.00 | 0.00 | 0.00 | Not | Excellent | Excellent | Fair | Good |
| | | | | | solidified | | | | |
| Example 42 | 15.00 | 0.00 | 0.00 | 0.70 | Not | Good | Good | Excellent | Excellent |
| | | | | | solidified | | | | |
| Example 43 | 15.00 | 0.00 | 0.00 | 0.50 | Not | Good | Good | Good | Excellent |
| | | | | | solidified | | | | |
| Example 44 | 15.00 | 0.00 | 0.00 | 0.30 | Not | Good | Good | Good | Excellent |
| | | | | | solidified | | | | |
| Example 45 | 15.00 | 0.00 | 0.00 | 0.10 | Not | Good | Good | Good | Excellent |
| | | | | | solidified | | | | |
| Example 46 | 15.00 | 0.00 | 0.00 | 0.00 | Not | Good | Good | Excellent | Excellent |
| | | | | | solidified | | | (+) | (+) |

TABLE 5

| Dispersing liquid | | |
|---|---|---|
| | Dispersing aid | Lubricant |

| | Silica | | | | Gly- | Propylene | Sorbitan | Decamethyl | Vinyl | |
|---|---|---|---|---|---|---|---|---|---|---|
| | | Content | Ethanol | Water | cerin | glycol | sesqui- | cyclopenta- | dimethi- | Hydrogenated |
| | | (mass | (mass | (mass | (mass | (PG) | oleate | siloxane | cone | polyisobutene |
| | Trade name | %) | %) | %) | %) | (mass %) | (mass %) | (mass %) | (mass %) | (mass %) |
| Ref. Ex. 1 | SUNSPHERE | 1.00 | 92.81 | 6.19 | 0.00 | 0.00 | 0.00 | 0.00 | 0.00 | 0.00 |
| | NP30 | | | | | | | | | |
| Comp. Ex. 1 | AEROSIL R812 | 1.00 | 55.31 | 23.69 | 0.00 | 0.00 | 0.00 | 0.00 | 0.00 | 20.00 |
| Comp. Ex. 2 | AEROSIL R812 | 1.00 | 45.94 | 34.06 | 0.00 | 0.00 | 0.00 | 19.00 | 0.00 | 0.00 |
| Comp. Ex. 3 | AEROSIL R812 | 1.00 | 52.05 | 23.45 | 2.50 | 6.00 | 0.00 | 15.00 | 0.00 | 0.00 |
| Comp. Ex. 4 | AEROSIL R812 | 1.00 | 58.59 | 11.91 | 0.00 | 0.00 | 5.00 | 23.50 | 0.00 | 0.00 |
| Comp. Ex. 5 | AEROSIL R812 | 1.00 | 58.61 | 11.89 | 2.50 | 6.00 | 5.00 | 15.00 | 0.00 | 0.00 |

| | Washing aid | Evaluation of performance | | | | |
|---|---|---|---|---|---|---|
| | Ethoxy | Solidified | Standing | Setting | | |
| | diglycol | or not | degree of | power of | Combing | Removal |
| | (mass %) | solidified | hair | hair style | ease | by rinsing |
| Ref. Ex. 1 | 0.00 | Not solidified | Fair (−) | Fair (−) | Excellent | Fair |
| Comp. Ex. 1 | 0.00 | Solidified | Unevaluable | Unevaluable | Unevaluable | Fair |
| Comp. Ex. 2 | 0.00 | Solidified | Unevaluable | Unevaluable | Unevaluable | Fair |
| Comp. Ex. 3 | 0.00 | Solidified | Unevaluable | Unevaluable | Unevaluable | Fair |
| Comp. Ex. 4 | 0.00 | Solidified | Unevaluable | Unevaluable | Unevaluable | Fair |
| Comp. Ex. 5 | 0.00 | Solidified | Unevaluable | Unevaluable | Unevaluable | Fair |

### (2) Observation of Solidification

The hairstyling agent compositions obtained in Examples 1 to 46, Referential Example 1, and Comparative Examples 1 to 5 were observed whether the whole amount of them solidified or not. The results are shown in Tables 1 to 5.

From the hairstyling agent compositions obtained in Examples 27 to 32 and Examples 35 to 41, precipitation of an aggregate of the hydrophobic silica and turbidity of the dispersing liquid were observed, but the hairstyling agent compositions did not entirely solidify. The hairstyling agent compositions obtained in Comparative Examples 1 to 5, on the other hand, entirely solidified due to gelation of the hydrophobic silica.

### (3) Application of Hairstyling Agent Composition to Hair

The hairstyling agent compositions obtained in Examples 1 to 46 and Referential Example 1 were each applied to hair by using a spray. With regards to each of the hairstyling agent compositions obtained in Comparative Examples 1 to 5, solidification of the whole hairstyling agent composition prevented application of it to the hair by using a spray.

### (4) Evaluation of Standing Degree

After application of any one of the hairstyling agent compositions obtained in Examples 1 to 46 and Referential Example 1 to the hair by using the above-mentioned method, hair strands were bundled and made to stand. The standing degree was evaluated based on the below-described criteria and results are shown in Tables 1 to 5.

A bundle of hair strands was formed and it was stroked upward from the root. When the standing state of the hair bundle was maintained even after it was released from hands, the standing degree was evaluated as “excellent”.

A bundle of hair strands was formed and it was not only stroked upward from the root but also grasped for a few seconds after stroking. When the standing state of the hair bundle was maintained even after it was released from hands, the standing degree was evaluated as “good”.

Grasping a bundle of hair strands for a few seconds was not enough so that the bundle of hair strands was grasped for 30 seconds. When the standing state of the hair bundle was maintained even after it was released from hands, the standing degree was evaluated as “fair”.

When a bundle of hair strands was grasped for from 30 to 60 seconds and at last the standing state of the hair bundle was maintained even after it was released from hands, the standing degree was evaluated as “fair (−)”.

It is to be noted that the hairstyling agent compositions obtained in Examples 1 to 5 were evaluated as “not evaluable” because they could not be applied to the hair by using a spray.

### (5) Evaluation of Hairstyle Setting Power

Hairstyle setting power of the hairstyling agent compositions obtained in Examples 1 to 46 and Referential Example 1 was evaluated. Evaluation results of the hairstyle setting power based on the below-described standards are shown in Table 1 to 5

When, after a bundle of hair strands was made to stand, it was retained firmly as was for long hours, the hairstyle setting power was evaluated as “excellent (+)”.

When, after a bundle of hair strands was made to stand, it was retained firmly as was, the hairstyle setting power was evaluated as “excellent”.

When, after a bundle of hair strands was made to stand, it was retained as was in a good condition, the hairstyle setting power was evaluated as “good”.

When, after a bundle of hair strands was made to stand, it was retained as was in a fair condition, the hairstyle setting power was evaluated as “fair”.

When, after a bundle of hair strands was made to stand, it was barely retained as was, the hairstyle setting power was evaluated as “fair (−)”.

### (6) Evaluation of Combing Ease

Combing ease of the hairstyling agent compositions obtained in Examples 1 to 46 and Referential Example 1 was evaluated by combing, after a bundle of hair strands was made to stand, the hair bundle from the root to the tip. Evaluation results of combing ease based on the below-described criteria are shown in Tables 1 to 5.

Combing ease was evaluated as “excellent (+)” when the hair bundle was combed completely smoothly from the root to the tip without being tangled in a comb.

Combing ease was evaluated as “excellent” when the hair bundle was combed from the root to the tip without being tangled in a comb.

Combing ease was evaluated as “good” when the hair bundle was combed from the root to the tip while being tangled in a comb only several times.

Combing ease was evaluated as “fair” when the hair bundle was combed from the root to the tip while always being tangled in a comb.

Combing ease was evaluated as “bad” when the hair bundle was not combed from the root to the tip.

### (7) Evaluation of Removal by Rinsing

The removal, by rinsing, of the hairstyling agent compositions obtained in Examples 1 to 46 and Referential Example 1 was evaluated by rinsing the compositions with a commercially available shampoo (“Merit”, trade name; product of Kao Corporation) after a bundle of hair strands was made to stand by the method described above in (4) in evaluation of standing degree. Evaluation results of the removal by rinsing based on the below-described criteria are shown in Tables 1 to 5.

The removal by rinsing was evaluated as “excellent (+)” when the hair was combed smoothly with fingers even before shampooing and after shampooing, no silica remained on the hair and therefore the hair provided no stiff feeling.

The removal by rinsing was evaluated as “excellent”, when the hair was combed smoothly with fingers during shampooing and after shampooing, almost no silica remained on the hair and therefore the hair provided almost no stiff feeling.

The removal by rinsing was evaluated as “good” when the hair was combed smoothly with fingers during shampooing and after shampooing, not so much silica remained on the hair and therefore the hair provided a little stiff feeling.

The removal by rinsing was evaluated as “fair” when the hair was finally combed with fingers during shampooing and silica was removed from the hair by shampooing but the shampooed hair provided a certain level of stiff feeling.

The removal by rinsing was evaluated as “bad” when the hair was not combed with fingers during shampooing and even after shampooing, much silica remained on the hair and the shampooed hair provided a stiff feeling.

### INDUSTRIAL APPLICABILITY

The present invention is usable as a hairstyling agent composition.

1. A hairstyling agent composition comprising a hydrophobic silica and a dispersing liquid for dispersing therein the hydrophobic silica.

2. The hairstyling agent composition according to claim 1, wherein the hydrophobic silica has an average particle size of from 0.1 nm to 20 μm.

3. The hair styling agent composition according to claim 1, wherein the dispersing liquid contains a lower alcohol.

4. The hairstyling agent composition according to claim 1, wherein the dispersing liquid contains a dispersing aid for enhancing the dispersibility of the hydrophobic silica in the dispersing liquid.

5. The hairstyling agent composition according to claim 4, wherein the dispersing aid is composed of a polyhydric alcohol and/or a surfactant.

6. The hairstyling agent composition according to claim 5, wherein the polyhydric alcohol contains at least one selected from a group consisting of dihydric alcohols, condensates of dihydric alcohols, trihydric alcohols, condensates of trihydric alcohols, tetrahydric alcohols, condensates of tetrahydric alcohols, polysaccharides, and compounds of polysaccharides.

7. The hairstyling agent composition according to claim 1, further comprising a lubricant.

8. The hairstyling agent composition according to claim 7, wherein the lubricant contains at least one selected from a group consisting of a silicone oil, a silicone powder, and hydrogenated polyisobutene.

9. The hairstyling agent composition according to claim 8, wherein the silicone oil contains at least one selected from a group consisting of dimethyl silicone oil, methyl phenyl silicone oil, cyclic dimethyl silicone oil, methyl hydrogen silicone oil, vinyl dimethicone, dimethylpolysiloxane, methyl phenyl polysiloxane, and polyorganosilsesquioxane.

10. The hairstyling agent composition according to claim 8, wherein the silicone powder contains at least one selected from a group consisting of trimethylsiloxysilicic acid, methylsiloxane network polymer, crosslinked silicone-network type silicone block copolymer, and crosslinked silicone powder.

11. The hairstyling agent composition according to claim 1, further comprising a washing aid.

12. The hairstyling agent composition according to claim 11, wherein the washing aid contains at least one selected from a group consisting of ethoxy diglycol, ethoxy ethanol, butoxy ethanol, and methoxy ethanol.

13. The hairstyling agent composition according to claim 1, further comprising a hair growth promoting component.

14. The hairstyling agent composition according to claim 13, wherein the hair growth promoting component contains at least one selected from a group consisting of β-glycyrrhetinic acid, dipotassium glycyrrhizate, monoammonium glycyrrhizate, alkaloid from Stephania cepharantha HAYATA (cepharanthine), swertia japonica extract, tinctura capsici, hinokitiol, iodized garlic extract, pyridoxine hydrochloride (Vitamin B6), dl-α-tocopherol (Vitamin E), dl-α-tocopherol acetate (Vitamin E), nicotinic acid amide, benzyl nicotinate, calcium pantothenate, D-pantothenyl alcohol, acetyl pantothenyl ethyl ether, biotin, adenosine, allantoin, isopropyl methylphenol, estradiol, ethinyl estradiol, capronium chloride, benzalkonium chloride, diphenhydramine hydrochloride. Kankohso 301, camphor, salicylic acid, nonanoic acid vanilamide, piroctone olamine, glyceryl pentadecanoate, l-menthol, mononitroguaiacol, resorcin, Sophora Angustifolia root extract, minoxidil, γ-aminobutyric acid, aloe extract, ginkgo extract, benzethonium chloride, mexiletine hydrochloride, Scutellaria Baicalensis root extract, auxin, oxendolone, hypericum extract, seaweed extract, cantharides tincture, diisopropylamine dichloroacetate, cyclosporin, ginger tincture, zinc pyrithione, lansium domesticum extract, calendula extract, cordyceps extract, 2-L-ascorbic acid dl-α-tocopherol phosphoric acid diester potassium salt, peppermint oil, 10-hydroxy-δ-2-decenoic acid, hydrocortisone, rosa rugosa var. pleva extract, mini-sasanishiki extract, reishi mushroom extract, polyoxyethylene (20) sorbitan monostearate, rehmanniae radix extract, placenta extract, soybean extract, belamcanda chinensis root extract, chlorella vulgaris extract, hematin porcine, mucopolysaccharide (porcine), Japanese pepper extract, clove extract, cherry leaf extract, sage extract, tilia cordata flower extract, tree paeony extract, and houttuynia cordata extract.

15. The hairstyling agent composition according to claim 13, wherein the hair growth promoting component is an oil soluble substance.
